(19) United States
(12) Patent Application Publication
(10) Pub. No.: US 2013/0116558 A1
(43) Pub. Date: May 9, 2013

(54) SYSTEM AND METHOD FOR DIAGNOSING AND TREATING FORMER PATIENTS

(71) Applicant: Steven Cogswell, Waterford, MI (US)

(72) Inventor: Steven Cogswell, Waterford, MI (US)

(21) Appl. No.: 13/290,660

(22) Filed: Nov. 7, 2011

(51) Int. Cl. A61B 8/00; A61M 31/00; G06Q 50/22

(52) U.S. Cl. 600/437; 705/2; 604/48

(57) ABSTRACT

A system and method diagnoses and treats a former patient of a healthcare facility such that the system and method prevents re-admission of the patient to the healthcare facility and admission of the patient to another healthcare facility. The patient experiences at least one health-related problem following original discharge of the patient from the facility and calls for remote assistance for resolving the problem at a residence of the patient. The system and method comprises steps of attempting to remotely resolve the problem, determining whether the problem has been remotely resolved, activating a response unit of clinicians to the residence if it is determined that the problem has not been remotely resolved, responding by the response unit to the residence, evaluating the patient, providing stabilizing treatment to the patient while the patient remains at the residence, determining whether the patient is stabilized such that the system and method prevents re-admission of the patient to the healthcare facility or admission of the patient to another healthcare facility, and re-evaluating the patient for appropriate transportation of the patient to and placement of the patient in the healthcare facility or other healthcare facility if it is determined that the patient is not stabilized.

### BACKGROUND OF INVENTION

1. Field of Invention

The invention relates, generally, to a system and method for diagnosing and treating a former patient and, more particularly, to such a system and method that prevents re-admission of such patient to a healthcare facility.

2. Description of Related Art

Generally speaking, a patient of a healthcare facility—such as a hospital—is discharged from the hospital shortly after he/she has sufficiently recovered from a health-related condition (or problem or difficulty)—such as an acute illness—treatment for which he/she was originally admitted to the hospital. (If the patient is “high risk,” the hospital generally identifies the patient as such.) If, after the discharge, the former patient becomes acutely ill again or distressed at, say, his/her residence (which frequently occurs at night when outpatient facilities are closed), the former patient calls local emergency-medical services for health-related assistance and may even call a healthcare provider—like a primary-care physician—of the former patient of the acute illness or distress. An emergency-medical-service unit (EMS unit) is then typically dispatched to the residence.

Yet, diagnostic and clinical- and medical-decision-making capabilities of the EMS unit are limited. For example, such diagnostic capability consists primarily of that related to taking the former patient's vital signs, i.e., blood pressure, pulse, respiration, pulse oximetry, and electrocardiogram (EKG). Because of these limitations, the EMS unit minimally stabilizes the former patient and then frequently must transport to the emergency room of the hospital him/her for further diagnosis and treatment thereof, which often results in his/her readmission to the hospital. Even when the former patient or EMS team makes contact with the primary-care physician, the only viable option is to return him/her to the hospital, in general, and ER thereof, in particular. In this context, the EMS unit is designed essentially as a unit primarily responsible for minimally stabilizing the former patient and then transporting him/her back to the hospital. Not only does such readmission fail the former patient and hospital, but it greatly increases cost to the healthcare industry, especially Medicare and Medicaid, as a whole.

Thus, there is a need for a system and method for diagnosing and treating former patients that does not fail the patients and hospitals and greatly increase cost to the healthcare industry, especially Medicare and Medicaid, as a whole. More specifically, there is a need for such a system and method that dispatches to a residence of a former patient a response unit of clinicians (such as paramedics and nurse practitioners and/or physician assistants) that can appropriately diagnose, stabilize, and treat the former patient while keeping the former patient at the residence. There is a need for such a system and method that also has “on board” diagnostic and clinical- and medical-decision-making capabilities. There is a need for such a system and method that also uses advanced diagnostic capabilities, such as x-ray laboratory studies and ultrasound. There is a need for such a system and method that also avoids unnecessary transport of the former patient back to the hospital and costs to re-admit him/her thereto.

### SUMMARY OF INVENTION

The invention overcomes the disadvantages in the related art in a system and method for diagnosing and treating a former patient of a healthcare facility such that the system and method prevents re-admission of the patient to the healthcare facility and admission of the patient to another healthcare facility. The patient experiences at least one health-related problem following original discharge of the patient from the facility and calls for remote assistance for resolving the problem at a residence of the patient. The system and method includes steps of attempting to remotely resolve the problem, determining whether the problem has been remotely resolved, activating a response unit of clinicians to the residence if it is determined that the problem has not been remotely resolved, responding by the response unit to the residence, evaluating the patient, providing stabilizing treatment to the patient while the patient remains at the residence, determining whether the patient is stabilized such that the system and method prevents re-admission of the patient to the healthcare facility or admission of the patient to another healthcare facility, and re-evaluating the patient for appropriate transportation of the patient to and placement of the patient in the healthcare facility or other healthcare facility if it is determined that the patient is not stabilized.

One advantage of the system and method for diagnosing and treating a former patient of the invention is that it does not fail the patients and hospitals and greatly increase cost to the healthcare industry, especially Medicare and Medicaid, as a whole.

Another advantage of the system and method for diagnosing and treating a former patient of the invention is that it dispatches to a residence of a former patient a response unit of clinicians (such as paramedics and nurse practitioners and/or physician assistants) that can appropriately diagnose, stabilize, and treat the former patient while keeping the former patient at the residence.

Another advantage of the system and method for diagnosing and treating a former patient of the invention is that it has “on board” diagnostic and clinical- and medical-decision-making capabilities.

Another advantage of the system and method for diagnosing and treating a former patient of the invention is that it uses advanced diagnostic capabilities, such as x-ray laboratory studies and ultrasound

Another advantage of the system and method for diagnosing and treating a former patient of the invention is that it avoids unnecessary transport of the former patient back to the hospital and costs to re-admit him/her thereto.

Other objects, features, and advantages of the system and method for diagnosing and treating a former patient of the invention are readily appreciated as the system and method becomes more understood while the subsequent detailed description of at least one embodiment of the system and method is read taken in conjunction with the accompanying drawing thereof.

### BRIEF DESCRIPTION OF FIGURE OF DRAWING

FIG. 1 is a schematic diagram depicting the steps of respective embodiments of the system and method for diagnosing and treating a former patient of the invention.

### DETAILED DESCRIPTION OF EMBODIMENTS OF INVENTION

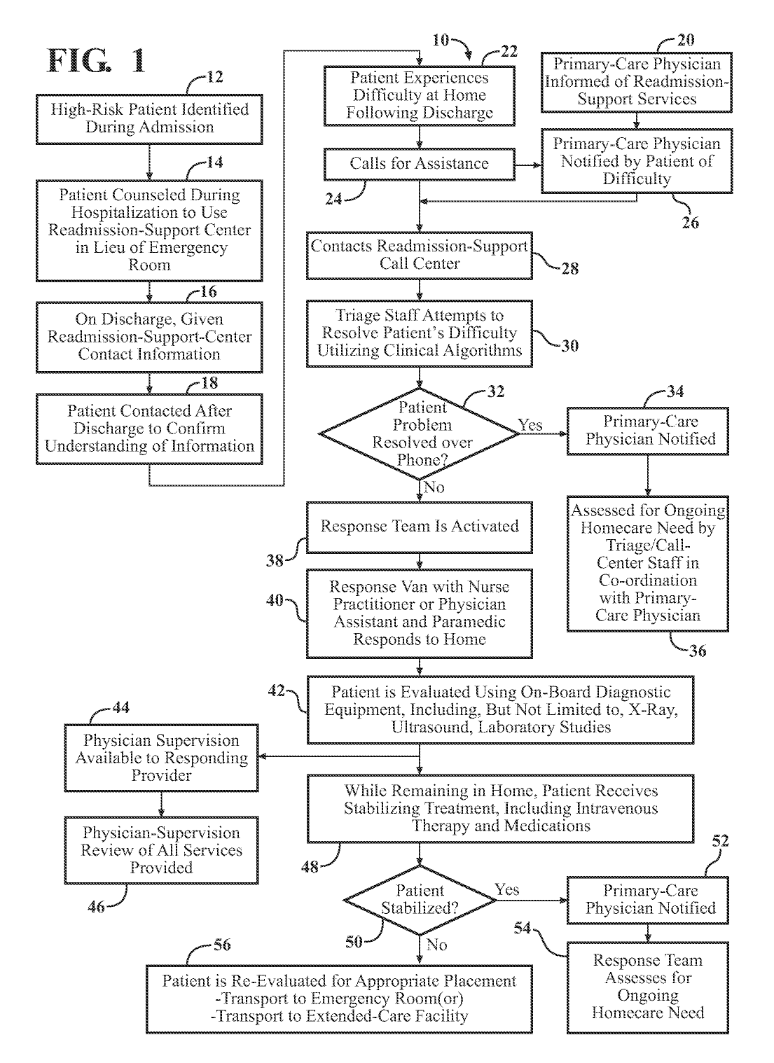

Referring now to FIG. 1, a system and method for diagnosing and treating a former patient of a healthcare facility (such that the system and method prevents re-admission of the patient to the healthcare facility and admission of the patient to another healthcare facility) of the invention is generally indicated at 10. In the system and method 10, the patient experiences at least one health-related problem following original discharge of the patient from the facility and calls for remote assistance for resolving the problem at a residence (or home) of the patient. FIG. 1 uses a flow diagram to illustrate the steps in a sequence of operations of respective embodiments of the system and method 10.

It should be appreciated by those having ordinary skill in the related art that the calling for remote assistance can be by telephoning or any other suitable method of calling—such as, but by no means limited to, e-mailing, telefaxing, or texting. It should be so appreciated also that, although the system and method 10 is designed to be employed especially in connection with the residence of the patient, the system and method 10 can be employed with any suitable type of location of the patient that is remote with respect to the hospital or other healthcare facility.

As shown in the FIGURE, the system and method 10 includes, in general, the steps of attempting to remotely resolve the problem 30, determining whether the problem has been remotely resolved 32, activating a response unit of clinicians to the residence if it is determined that the problem has not been remotely resolved 38, responding by the response unit to the residence 40, evaluating the patient 42, providing stabilizing treatment to the patient while the patient remains at the residence 48, determining whether the patient is stabilized such that the system and method 10 prevents re-admission of the patient to the healthcare facility or admission of the patient to another healthcare facility 50, and re-evaluating the patient for appropriate transportation of the patient to and placement of the patient in the healthcare facility or other healthcare facility if it is determined that the patient is not stabilized 56.

In an embodiment of the system and method 10, the patient is “high risk,” and the healthcare facility—such as a hospital—identifies the patient as such during original admission of the patient to the hospital 12. During the hospitalization, the hospital counsels the patient for the patient to use the system and method 10 in lieu of services of an emergency room if the patient experiences the health-related problem—such as an acute illness (treatment for which he/she was originally admitted to the hospital as a “high risk” patient)—following the original discharge 14. Also, a healthcare provider, like a primary-care physician, of the patient is informed of the system and method 10 at any time during or after the hospitalization 20.

More specifically, the hospital counsels the patient for him/her to use services of, for example, a “re-admission support” center of the hospital in lieu of those of an emergency room thereof if the patient becomes acutely ill again or distressed after he/she is originally discharged from the hospital 14. Then, upon the patient's being discharged from the hospital shortly after he/she has sufficiently recovered from the acute illness, the hospital provides the former patient with “contact” information of the “re-admission support” center 16. After the discharge, the hospital contacts the former patient for the former to confirm with the latter his/her understanding of the information 18. Meanwhile, the primary-care physician is informed of the services by, for instance, the hospital 20. If, after the discharge, the former patient, in fact, becomes acutely ill again or distressed at, say, his/her residence (which frequently occurs at night when outpatient facilities are closed) 22, the former patient calls the “re-admission support” center of the hospital for him/her to use the services thereof (in lieu of services of an emergency room of the hospital) 28 and may even notify the primary-care physician of the acute illness or distress 26.

It should be appreciated by those having ordinary skill in the related art that, although the system and method 10 is designed to be employed especially with a patient who is “high risk,” the system and method 10 can be employed with any suitable type of patient, including one that is not “high risk.” It should be so appreciated also that, although the healthcare facility is described herein and in the drawing as a hospital, the healthcare facility can be any suitable type of one. It should be so appreciated also that the emergency room can be any suitable part of any suitable facility designed and used for treatment of people requiring immediate health-related attention. It should be so appreciated also that, although the health-related problem is described herein as an acute illness, the health-related problem can be any suitable type of one. It should be so appreciated also that, although the healthcare provider is described herein as the primary-care physician, the healthcare provider can be any suitable type of one. It should be so appreciated also that the “re-admission support” center can be any suitable entity that offers services to a formerly admitted patient of the entity related to possible re-admission of the patient to the entity.

In an embodiment of the system and method 10, the acute illness is attempted to be treated over a telephone by a member or members of a triage staff of the hospital or another healthcare facility 30. In particular, the triage staff uses clinical algorithms for it to attempt to treat the acute illness 30. However, it should be appreciated by those having ordinary skill in the related art that any suitable type of staff can attempt to treat the acute illness. It should be so appreciated also that the triage staff can use any suitable means for it to attempt to treat the acute illness.

In an embodiment of the system and method 10, the primary-care physician is notified of the acute illness and treatment if it is determined that the acute illness has been remotely treated 34. Then, need of the patient for ongoing home-care is assessed 36. In particular, the need is assessed by the triage staff and/or a call-center staff of the hospital in coordination with the primary-care physician 36. However, those having ordinary skill in the related art should appreciate that the need can be assessed by any suitable staff or suitable staffs of the hospital.

If, on the other hand, it is determined that the acute illness has not been remotely treated 34, then, as described above, the response team of clinicians is activated to the residence 38. In an embodiment of the system and method 10, the clinicians include a paramedic and a nurse practitioner and/or physician assistant 40, and the patient is evaluated using diagnostic equipment 42. The response unit responds to the residence via a motor vehicle (for instance, a van) 40, and the diagnostic equipment is on-board the vehicle 42. In particular, the patient is evaluated using x-ray, ultrasound, and/or laboratory studies 42.

However, it should be appreciated by those having ordinary skill in the related art that the response unit can include any suitable number and type of healthcare personnel. It should be so appreciated also that the patient can be evaluated by any suitable means. It should be so appreciated also that the response unit can respond to the residence via any suitable means. It should be so appreciated also that the patient can be evaluated by any suitable means.

In an embodiment of the system and method 10, supervision is made available to the response unit 44. In particular, the supervision is by a physician 44. Also, all services provided by the response unit to the patient are supervisorily reviewed 46. In particular, the supervisory review is by a physician as well. However, those having ordinary skill in the related art should appreciate that the supervision and supervisory review can be by any suitable healthcare personnel.

In an embodiment of the system and method 10, the treatment includes intravenous therapy and/or medications 48. However, it should be appreciated by those having ordinary skill in the related art that the patient can be treated by any suitable means, in general, and the treatment can include any suitable type of therapy and/or medications, in particular.

In an embodiment of the system and method 10, the primary-care physician is notified of the stabilization if it is determined that the patient is stabilized 52. And, need of the patient for ongoing home-care is assessed 54. In particular, the need is assessed by the response unit 54. However, those having ordinary skill in the related art should appreciate that the need can be assessed by any suitable healthcare personnel, in general, and member of the response unit, in particular.

If, on the other hand, it is determined that the patient is not stabilized 52, then, as described above, the patient is re-evaluated for appropriate transportation of the patient to and placement of the patient in the healthcare facility or the other healthcare facility. In an embodiment of the system and method 10, the patient is re-evaluated for appropriate transportation of the patient to and placement of the patient in the emergency room and/or an extended-care facility 56. Those having ordinary skill in the related art should appreciate that the re-evaluation can be by any suitable healthcare personnel, in general, and member of the response unit, in particular.

The system and method 10 does not fail the patients and hospitals and greatly increase cost to the healthcare industry, especially Medicare and Medicaid, as a whole. Also, the system and method 10 dispatches to a residence of a former patient a response unit of clinicians (such as paramedics and nurse practitioners and/or physician assistants) that can appropriately diagnose, stabilize, and treat the former patient while keeping the former patient at the residence. Furthermore, the system and method 10 has “on board” diagnostic and clinical- and medical-decision-making capabilities. In addition, the system and method 10 uses advanced diagnostic capabilities, such as x-ray laboratory studies and ultrasound. Moreover, the system and method 10 avoids unnecessary transport of the former patient back to the hospital and costs to re-admit him/her thereto.

It should be appreciated by those having ordinary skill in the related art that the system and method 10 has been described above in an illustrative manner. It should be so appreciated also that the terminology that has been used above is intended to be in the nature of words of description rather than of limitation. It should be so appreciated also that many modifications and variations of the system and method 10 are possible in light of the above teachings. It should be so appreciated also that, within the scope of the appended claims, the system and method 10 may be practiced other than as specifically described above.

1. A system and method for diagnosing and treating a former patient of a healthcare facility such that the system and method prevents re-admission of the patient to the healthcare facility and admission of the patient to another healthcare facility, wherein the patient experiences at least one health-related problem following original discharge of the patient from the facility and calls for remote assistance for resolving the problem at a residence of the patient, said system and method comprising steps of:
attempting to remotely resolve the problem;
determining whether the problem has been remotely resolved;
activating a response unit of clinicians to the residence if it is determined that the problem has not been remotely resolved;
responding by the response unit to the residence;
evaluating the patient;
providing stabilizing treatment to the patient while the patient remains at the residence;
determining whether the patient is stabilized such that the system and method prevents re-admission of the patient to the healthcare facility and admission of the patient to another healthcare facility; and
re-evaluating the patient for appropriate transportation of the patient to and placement of the patient in at least one of the healthcare facility and the other healthcare facility if it is determined that the patient is not stabilized.

2. A system and method for diagnosing and treating a former patient as set forth in claim 1, wherein the system and method comprises further the steps of counseling the patient for the patient to use the system and method in lieu of services of an emergency room if the patient experiences the health-related problem following the original discharge and informing a primary-care physician of the patient of the system and method.

3. A system and method for diagnosing and treating a former patient as set forth in claim 1, wherein the problem is attempted to be remotely resolved by a triage staff of the healthcare facility or another healthcare facility.

4. A system and method for diagnosing and treating a former patient as set forth in claim 1, wherein the triage staff uses clinical algorithms for it to attempt to remotely resolve the problem.

5. A system and method for diagnosing and treating a former patient as set forth in claim 1, wherein the system and method comprises further the step of notifying a primary-care physician of the patient of the problem and resolution if it is determined that the problem has been remotely resolved.

6. A system and method for diagnosing and treating a former patient as set forth in claim 5, wherein the system and method comprises further the step of assessing need of the patient for ongoing home-care.

7. A system and method for diagnosing and treating a former patient as set forth in claim 6, wherein the need is assessed by at least one of a triage and call-center staff of the healthcare facility in coordination with the primary-care physician.

8. A system and method for diagnosing and treating a former patient as set forth in claim 1, wherein the response unit includes a paramedic and at least one of a nurse practitioner and physician assistant.

9. A system and method for diagnosing and treating a former patient as set forth in claim 1, wherein the patient is evaluated using diagnostic equipment.

10. A system and method for diagnosing and treating a former patient as set forth in claim 9, wherein the patient is evaluated using at least one of x-ray, ultrasound, and laboratory studies.

11. A system and method for diagnosing and treating a former patient as set forth in claim 9, wherein the response unit responds to the residence via a motor vehicle and the diagnostic equipment is on-board the vehicle.

12. A system and method for diagnosing and treating a former patient as set forth in claim 1, wherein the treatment includes at least one of intravenous therapy and medications.

13. A system and method for diagnosing and treating a former patient as set forth in claim 1, wherein the system and method comprises further the step of notifying a primary-care physician of the patient of the stabilization if it is determined that the patient is stabilized.

14. A system and method for diagnosing and treating a former patient as set forth in claim 13, wherein the system and method comprises further the step of assessing need of the patient for ongoing home-care.

15. A system and method for diagnosing and treating a former patient as set forth in claim 14, wherein the need is assessed by the response unit.

16. A system and method for diagnosing and treating a former patient as set forth in claim 1, wherein the patient is re-evaluated for appropriate transportation of the patient to and placement of the patient in at least one of an emergency room and extended-care facility.

17. A system and method for diagnosing and treating a former patient as set forth in claim 1, wherein the system and method comprises further the step of making supervision available to the response unit.

18. A system and method for diagnosing and treating a former patient as set forth in claim 17, wherein the supervision is by a physician.

19. A system and method for diagnosing and treating a former patient as set forth in claim 1, wherein the system and method comprises further the step of supervisorily reviewing all services provided by the response unit to the patient.

20. A system and method for diagnosing and treating a former patient as set forth in claim 19, wherein the supervisory review is by a physician.
